(19) United States
(12) Patent Application Publication
(10) Pub. No.: US 2004/0037812 A1
(43) Pub. Date: Feb. 26, 2004

(54) Injectable chondrocyte implant

(75) Inventors: Bruno M. Giannetti, Bonn (DE); Veronika Kunert, Recklinghausen (DE)

Correspondence Address: MORGAN, LEWIS & BOCKIUS LLP, 1701 MARKET STREET, PHILADELPHIA, PA, 19103-2921, US

(73) Assignee: Verigen AG

(21) Appl. No.: 10/427,463

(22) Filed: May 1, 2003

Related U.S. Application Data
(60) Provisional application No. 60/376,709, filed on May 1, 2002.

(51) Int. Cl. A61K 45/00

(52) U.S. Cl. 424/93.7; 424/426

(57) ABSTRACT

The present invention relates to a flowable implantable composition comprising a support material, such as various forms of collagen or alginate beads or threads, which can support the attachment and growth of chondrocyte cells thereto, ad a method of making a flowable implantable composition comprising a support material and chondrocytes retained thereon. Further, the present invention relates to a method for the effective treatment of articulating joint surface cartilage by the transplantation of a flowable composition including a support material and chondrocyte cells retained thereon.

### FIELD OF THE INVENTION

The present invention relates to the field of chondrocyte cell implantation, cartilage grafting, healing, joint repair and the prevention of arthritic pathologies. In particular, the present invention is directed to a new form of implant and to new methods for chondrocyte cell implantation and cartilage regeneration.

### BACKGROUND OF THE INVENTION

More than 500,000 arthroplastic procedures and total joint replacements are performed each year in the United States. Approximately the same numbers of similar procedures are performed in Europe. Included in these numbers are about 90,000 total knee replacements and around 50,000 procedures to repair defects in the knee per year (In: Praemer A., Furner S., Rice, D. P., Musculoskeletal conditions in the United States, Park Ridge, Ill.: American Academy of Orthopaedic Surgeons, 1992, 125). A method for regeneration-treatment of cartilage would be most useful, and could be performed at an earlier stage of a joint damage, thus reducing the number of patients needing artificial joint replacement surgery. With such preventative methods of treatment, the number of patients developing osteoarthritis would also decrease.

Techniques used for resurfacing the cartilage structure in damaged joints have mainly attempted to induce the repair of cartilage using subchondral drilling, abrasion and other methods whereby there is excision of diseased cartilage and subchondral bone, leaving vascularized cancellous bone exposed (Insall, J. Ficat R. P. et al, Clin Orthop. 1979,144,74; Johnson L. L., In: (McGinty J, B., Ed.)Operative Arthroscopy, New York: Raven Press, 1991, 341).

Coon and Cahn (1966, Science 153: 1116) described a technique for the cultivation of cartilage synthesizing cells from chick embryo somites. Later Cahn and Lasher (1967, PNAS USA 58: 1131) used the system for analysis of the involvement of DNA synthesis as a prerequisite for cartilage differentiation. Chondrocytes respond to both EGF and FGF by growth (Gospodarowicz and Mescher, 1977, J. Cell Physiology 93: 117), but ultimately lose their differentiated function (Benya et al., 1978, Cell 15: 1313). Methods for growing chondrocytes were described and are principally being used with minor adjustments as described by (Brittberg M. et al., New Engl. J. Med. 1994, 331, 889). Cells grown using these methods were used as autologous transplants into knee joints in patients.

International Application Number PCT/US00O/0654 1, assigned to Chondros, Inc. of Baltimore, Md., describes cells grown on a microcarrier. The cells are then separated from the microcarrier by enzymatic digestion. This reference also describes various polymers which can serve as scaffolds for cells to be used for implantation. The entire content of International Application Number PCT/US00/06541 is hereby incorporated by reference.

Additionally, Kolettas et al. examined the expression of cartilage-specific molecules such as collagens and proteoglycans under prolonged cell culturing. They found that despite morphological changes during culturing in monolayer cultures (Aulthouse, A. et al.,, In Vitro Cell Dev. Biol,, 1989,25,659; Archer, C. et al., J. Cell Sci. 1990, m97,361; Haanselmann, H. et al., J. Cell Sci. 1994,107,17; Bonaventure, J. et al., Exp. Cell Res. 1994,212,97) when compared to suspension cultures grown over agarose gels, alginate beads or as spinner cultures (retaining a round cell morphology) the expressed markers such as types II and IX collagens and the large aggregating proteoglycans, aggrecan, versican and link protein did not change. (Kolettas, E. et al., Science 1995,108,1991).

The articular chondrocytes are specialized mesenchymal derived cells found exclusively in cartilage. Cartilage is an avascular tissue whose physical properties depend on the extracellular matrix produced by the chondrocytes. During endochondral ossification chondrocytes undergo maturation leading to cellular hypertrophy, characterized by the onset of expression of type X collagen (Upholt, W. B. and Olsen, R. R., In: Cartilage Molecular Aspects (Hall, B. & Newman, S, Eds) CRC Boca Raton 1991, 43; Reichenberger, E. et al., Dev. Biol. 1991,148,562; Kirsch, T. et al., Differentiation, 1992,52,89; Stephens, M. et al., J. Cell Sci. 1993,103,1111).

### SUMMARY OF THE INVENTION

The present invention provides an implantable composition comprising a support material, preferably a solid or semi-solid material including microparticulate beads, threads, wafers, balls of thread, or a combination of beads, threads, wafers, and/or balls of thread (hereafter “microparticulate support material”). In one embodiment, the microparticulate support material is of varying size and shape. In one embodiment, the microparticulate support material supports the attachment and growth of chondrocyte cells or other types of cells thereto, and which in some embodiments with chondrocytes retained on the surface of the microparticulate support material, is flowable before and/or after injection into the site of implantation.

In an embodiment of the present invention, chondrocytes grow or adhere (hereafter collectively referred to as “adhere”) on the surface as well as in the microparticulate support material because the microparticulate support material has one or more porous openings in the surface. In use, the implantable composition of the present invention optionally further includes one or more of an adhesive and/or excipient, such as a gel, collagen, fibrin glue, autologous, semi-autologous and non-autologous glue as well as collagen gel, skin glues, surgical glues, and alginates.

The implantable composition according to the present invention can then be administered to a subject (typically by injection) as one or more of the following: 1) a mixture of adhesive and/or an excipient and the implantable composition, 2) a layer of the implantable composition, optionally including an adhesive or an excipient, followed by a layer of one or more adhesives, 3) a layer of one or more adhesives followed by a layer of the implantable composition, optionally including an adhesive or an excipient, or 4) a layer of the implantable composition free of adhesive or an excipient.

The invention also includes a method of making an implantable composition comprising a microparticulate support material and chondrocyte cells or other cells capable of forming cartilage or differentiating into cells that are capable of forming cartilage retained thereon. Other cells, such as mesenchymal cells, blood cells and fat cells, can be used in the present invention. In other embodiments, the present invention includes a method of making the implantable composition described above in combination with an adhesive or excipient. Further, the present invention provides a method for the effective treatment (for example, enhancing a patient's use of a damaged joint surface) of articulating joint surface cartilage by the implantation or transplantation of a composition including a microparticulate support material and chondrocyte cells retained thereon and/or therein optionally in combination with an adhesive or excipient.

As used herein, “about” means plus or minus approximately ten percent of the indicated value, such that “about 20 microns” indicates approximately 18 to 22 microns. The size of the particle can be determined by conventional methods known to those of skill in the art.

### BRIEF DESCRIPTION OF THE DRAWINGS

FIG. 1 is a side view of a portion of a thread of the present invention with cells adhered to and growing on its surface.

FIG. 2 is a side view of a bead, microsphere, or microbead of the present invention with cells adhered to and growing on its surface.

FIG. 3 is a graphical representation of the average number of cells harvested from tested support materials.

FIG. 4 is a graphical representation of the average viability of cells harvested from tested support materials.

FIG. 5 is a graphical representation of a heterogeneous collagen gel formed from packed microparticle beads of the present invention.

FIG. 6 is a graphical representation of a sponge-like material formed from threads of the present invention.

FIG. 7 is a graphical representation of a collagen sponge-like material of dried insoluble fibers packed within a three-dimensional volume.

FIG. 8 is a graphical representation of a homogenous collagen gel and a method of collagen gel formation.

FIG. 9 is a graphical representation of a collagen sponge-like material coated with a collagen film.

FIG. 10 is a graphical representation of an apparatus used to manufacture balls of thread of the present invention.

FIG. 11 is a graphical representation of a process for the manufacture of balls of thread of the present invention.

FIG. 12 is a graphical representation of a process for the manufacture of balls of thread of the present invention as well as balls of thread of the present invention.

FIG. 13 is a graphical representation of an apparatus used to manufacture beads of the present invention.

FIG. 14 is a graphical representation of a process for the manufacture of beads of the present invention.

FIG. 15 is a graphical representation of a process for the manufacture of beads of the present invention.

FIG. 16 is a graphical representation of an apparatus used to manufacture wafers of the present invention.

FIG. 17 is a graphical representation of a process for the manufacture of a wafer of the present invention.

FIG. 18 is a graphical representation of a process for the manufacture of wafer of the present invention.

FIG. 19 is a microscopic view of a ball of thread and a wafer, each formed from collagen thread of the present invention.

FIG. 20 is a microscopic view of collagen beads of the present invention.

FIG. 21 is a microscopic view of chondrocyte cells.

FIG. 22 is a microscopic view of chondocyte cells.

FIG. 23 is a microscopic view of chondrocyte cells.

### DETAILED DESCRIPTION OF THE INVENTION

The present invention includes an implantable composition comprising a microparticulate support material which can support the attachment and/or growth of chondrocyte cells on or in the microparticulate support material surface. The present invention further includes a method of making an implantable composition comprising a microparticulate support material with chondrocytes attached and/or grown thereon or therein. Further, the present invention includes a method for the effective treatment of damaged articulating joint surface cartilage by the transplantation or implantation of a composition including a microparticulate support material and chondrocyte cells attached and/or grown therein and/or thereon. The method for the effective treatment of articulating joint surface cartilage by implanting or transplanting a composition includes placing the composition upon the surface or within the area to be treated optionally by injection, particularly arthroscopic injection or another minimally invasive placement, and permitting the growth of the chondrocyte cells on the surface or within the area, thereby restoring cartilage tissue. In one embodiment, the method finds particular use in the treatment of joint surface cartilage in joints that have a minimal amount of space between bone surfaces, such as, but not limited to, ball and socket joints of the shoulder and hip, and other joints such as digital joints of the hands and feet and facial joints such as the jaw.

Additionally, in some embodiments of the composition and methods of the present invention, the present invention optionally includes the use of one or more biocompatible adhesives as well as one or more excipients. As used herein and described in more detail below, an excipient is a biocompatible material which can affect the flowability of the implantable composition. The adhesive is used to retain the composition of the present invention on a desired surface or in a desired area to be treated. In one embodiment, the adhesive is selected such that it functions as a hemostatic barrier and optionally also affect the flowability of the implantable composition before and after implantation in a manner similar to an excipient.

Each component of the present invention is discussed in more detail below. For the purposes of description only, collagen is described herein as an exemplary biodegradable material for use as threads, beads, balls of thread and/or wafers, although other biodegradable materials suitable for use in this invention may also be used.

### The Implantable Composition

The implantable composition of the present invention includes a microparticulate support material (also referred to as “support material” or “porous collagen biomaterial”) and cells adhered thereto, or therein if the surface of the support material is porous.

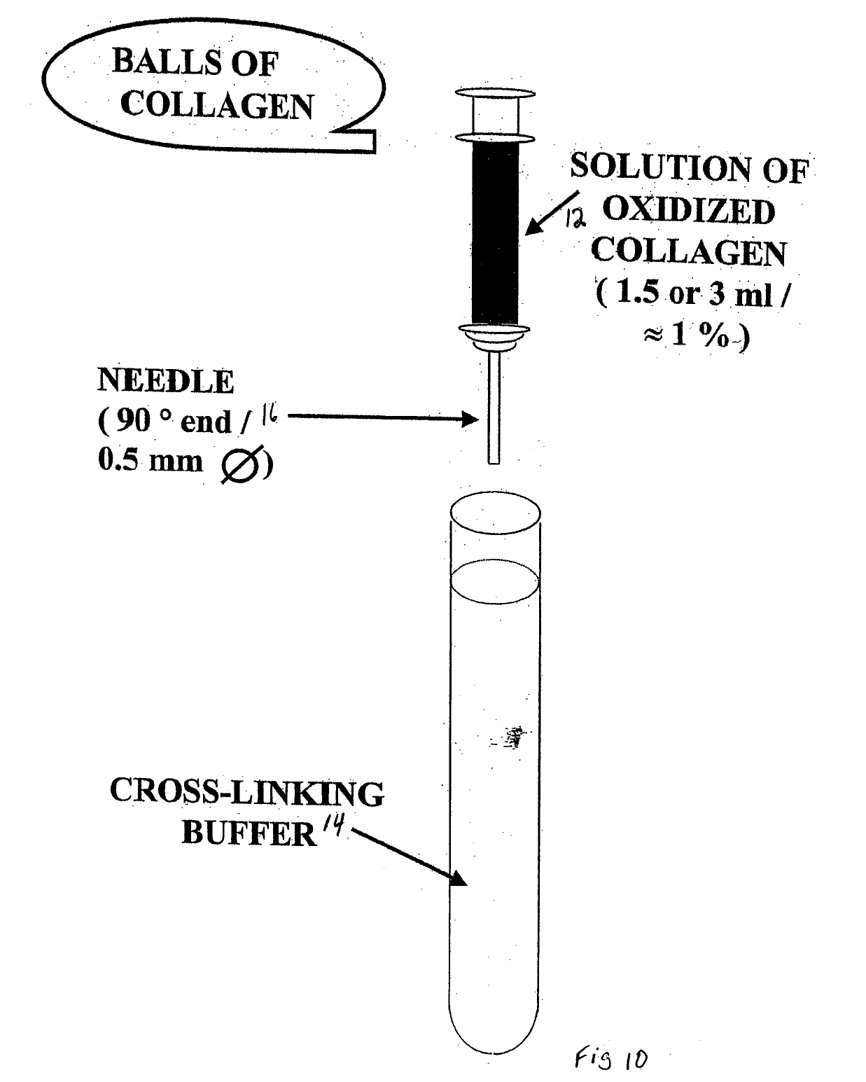

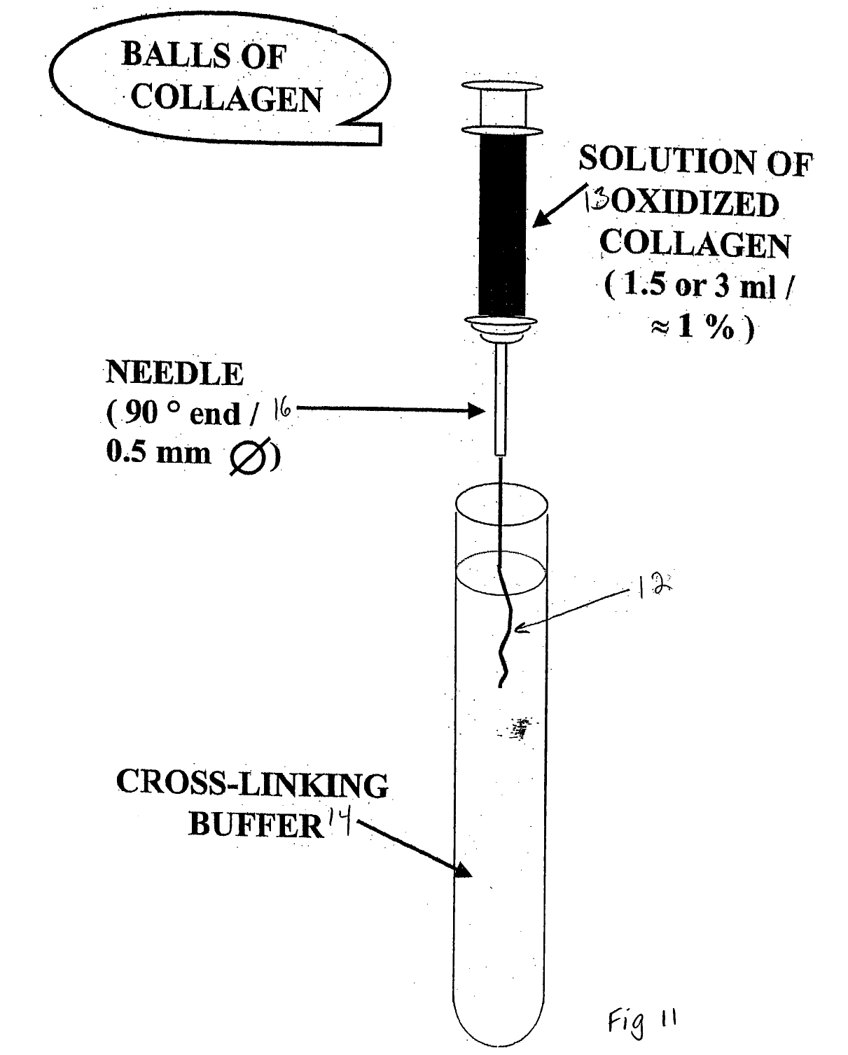

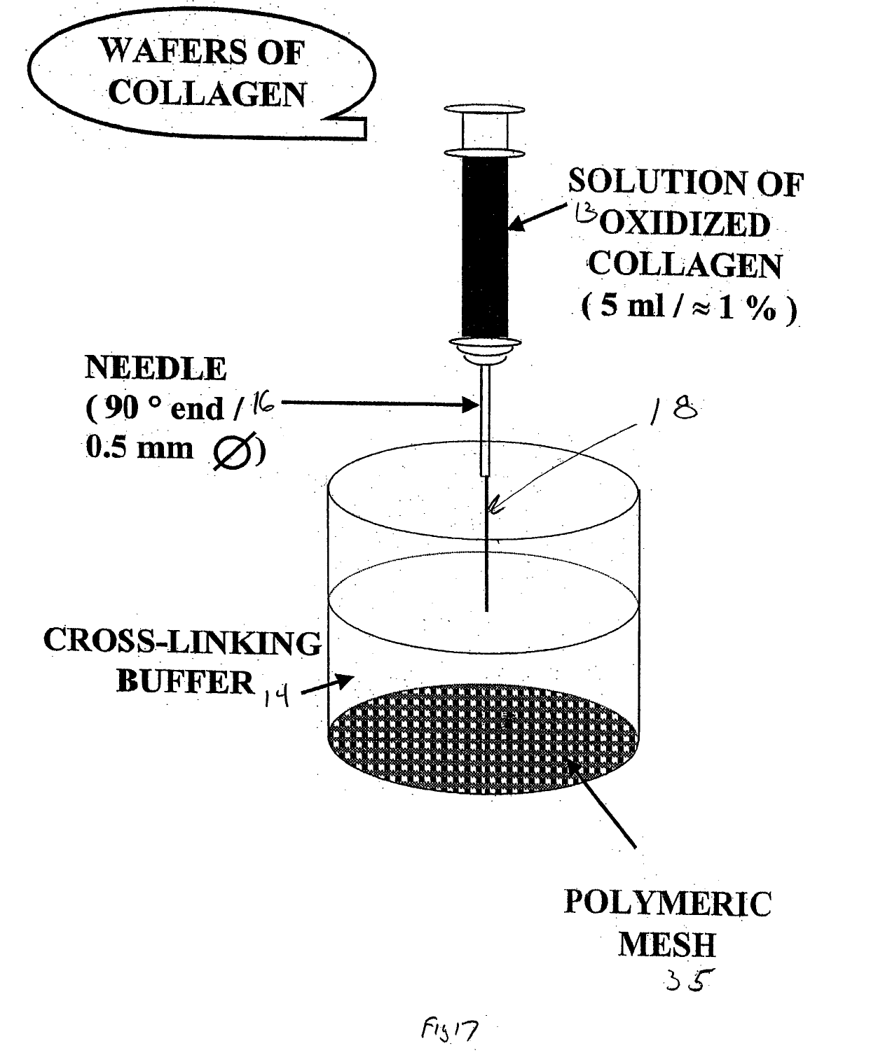

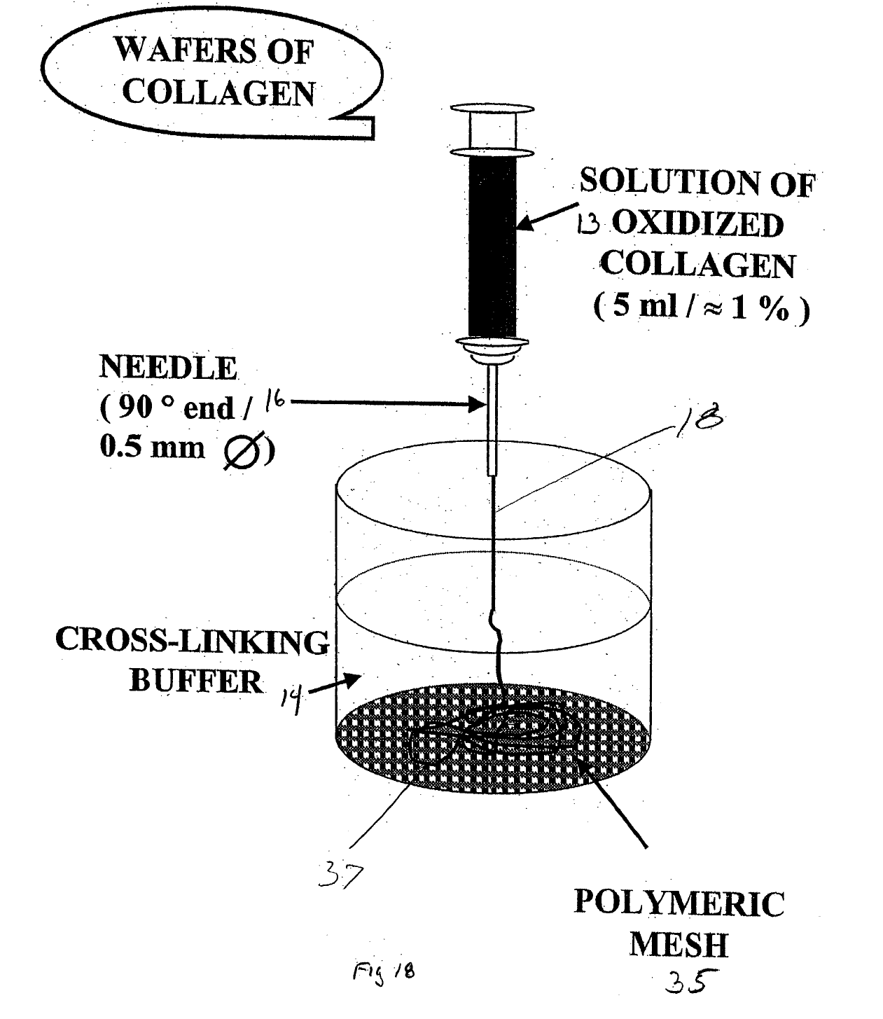

The microparticulate support material can be made by injecting an oxidized solution of collagen into a cross-linking bath (as described below with reference to FIGS. 10-18). In one such embodiment, the oxidized collagen is manufactured by using pepsin extraction techniques to form collagen fibers which are maintained at −20° C. Periodically, the fibers are subjected to acid oxidation to oxidize carbohydrate and hydroxylysine functional groups, which create aldehyde groups. The collagen can then be precipitated in a solution of NaCl and then washed with additional NaCl. After precipitation and washing, the precipitate is washed again with acetone to form an oxidized collagen powder, which can then be dissolved in acid to yield the oxidized collagen solution suitable for injection into a cross-linking bath, wherein a reaction between 1) the amine groups and 2) oxidized carbohydrate and hydroxylysine groups can occur, thereby forming a polyimine cross-linked network. In one embodiment, needle 16, having a 90 degree end and a 0.5 millimeter diameter, can be used to inject a 0.5-1.5% collagen solution into a cross-linking bath, as described in more detail below with reference to FIGS. 10-12.

To form porous collagen biomaterials, typically the collagen alpha-chains are covalently attached to fibrils via a cross-linking technique. Initially, the collagen is oxidized by periodic acid to generate aldehyde groups within the alpha-chain through oxidation of hydroxylysine and sugar residues. In one embodiment, the collagen can be cross-linked in a manner described by Tardy et al., U.S. Pat. No. 4,931,546, the entire content of which is hereby incorporated by reference. As described below, beads, threads, balls of thread, and wafers can then be formed by injecting the collagen gel through a capillary tube. Cross-linking occurs at a neutral pH by reaction of aldehyde groups (R—CHO) with amino groups (R′—NH₂) which in turn generates polyimine R—CH═N—R′ cross links, as described in U.S. Pat. No. 4,931,546. In such an embodiment, the threads may or may not be cross-linked with glutaraldehyde. In yet another embodiment, a portion of the collagen structure is coated with an additional amount of collagen to form a film on a portion of the surface of the structure. The film functions as a barrier to prevent cell migration.

The formation of collagen beads 22, threads 12, balls of thread 12, and wafers 37 involve different methods of injection of collagen into a cross-linking bath, and each method is described in more detail below with reference to FIGS. 10-18. In one embodiment, after injection into the bath, the acidity of the collagen support material is neutralized and subjected to glycerol incubation. In one embodiment, the support material can then be dried under air flow and sterilized via radiation, such as gamma sterilization, yielding a support material suitable for cell culturing.

As used herein a support material is of the form of thread 12, balls of thread 12 or beads 22, or wafer 37, and/or mixtures thereof.

A. Threads

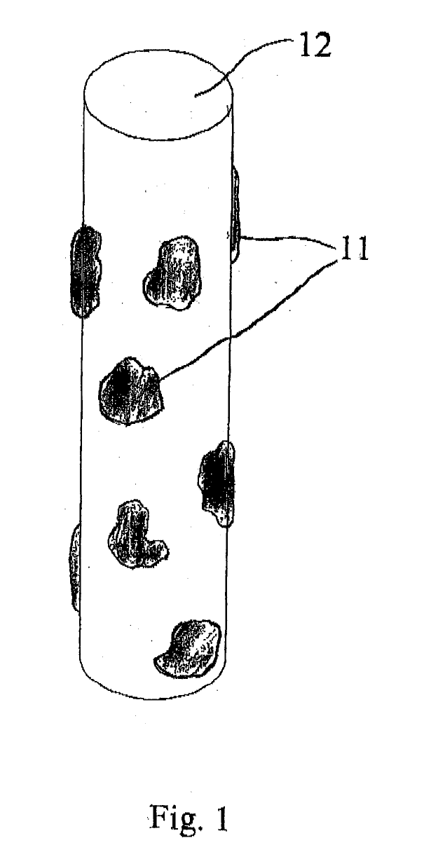

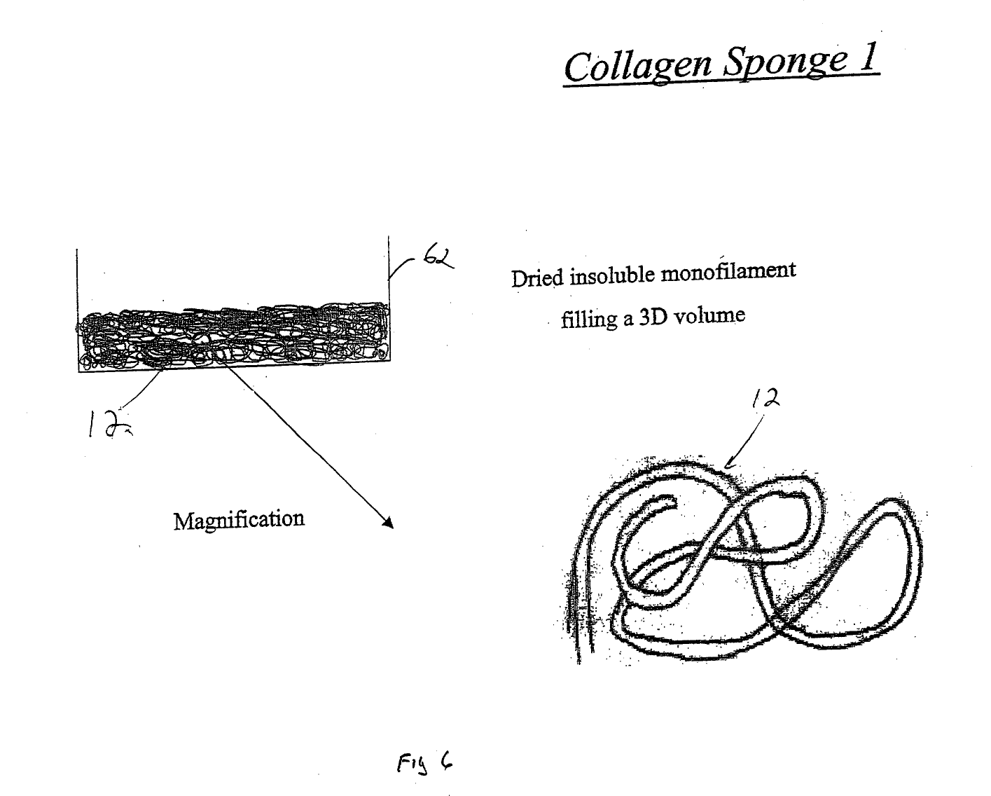

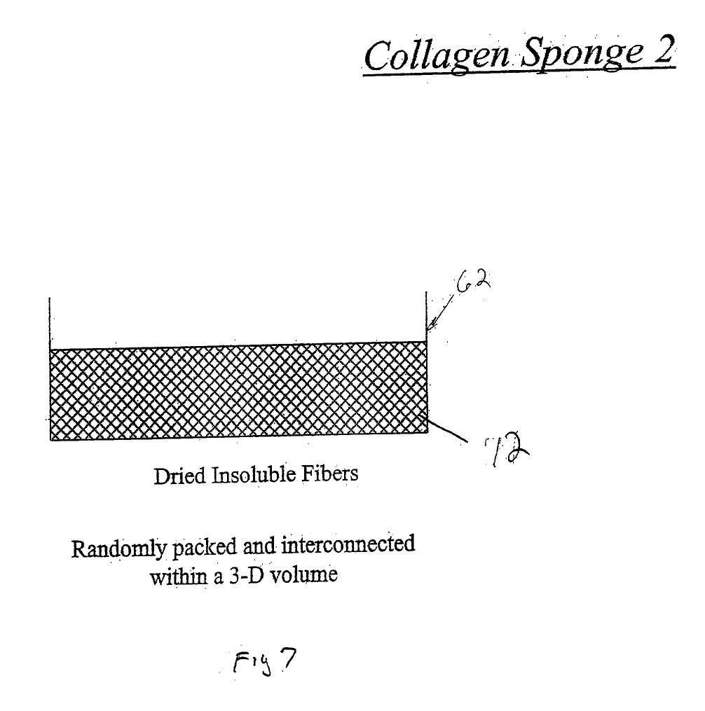

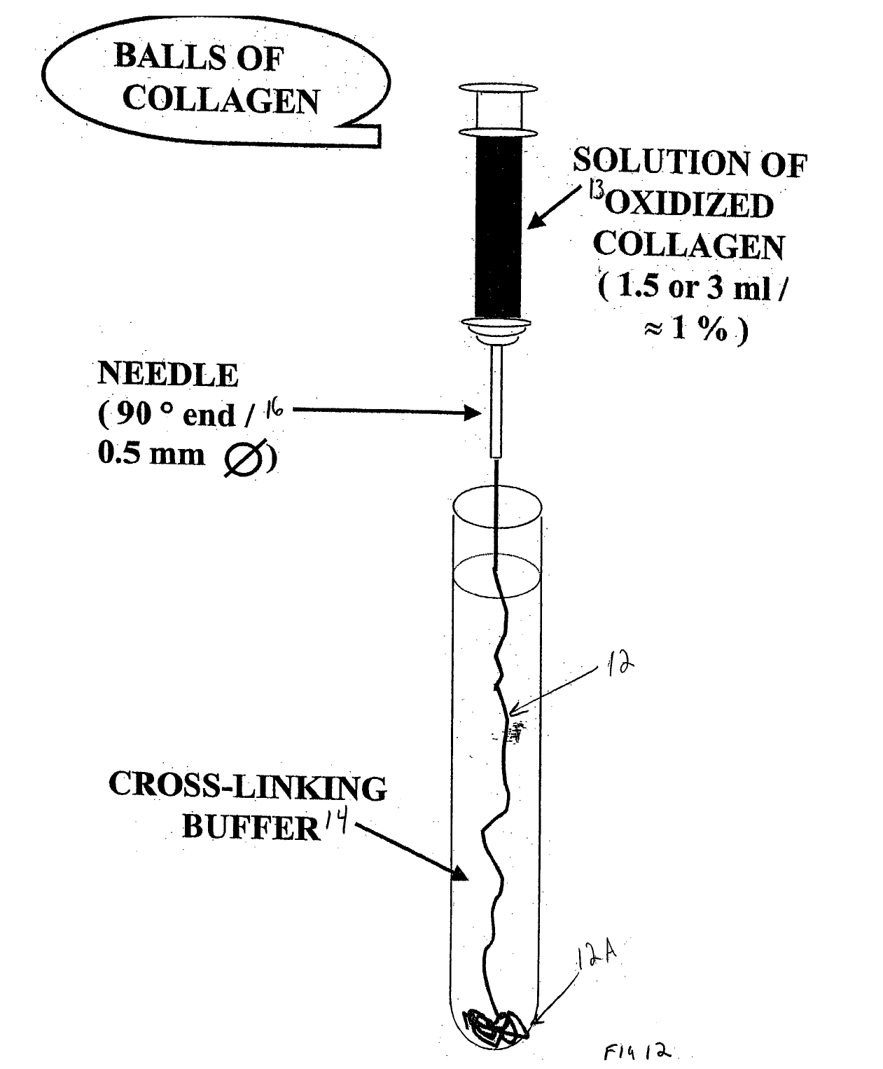

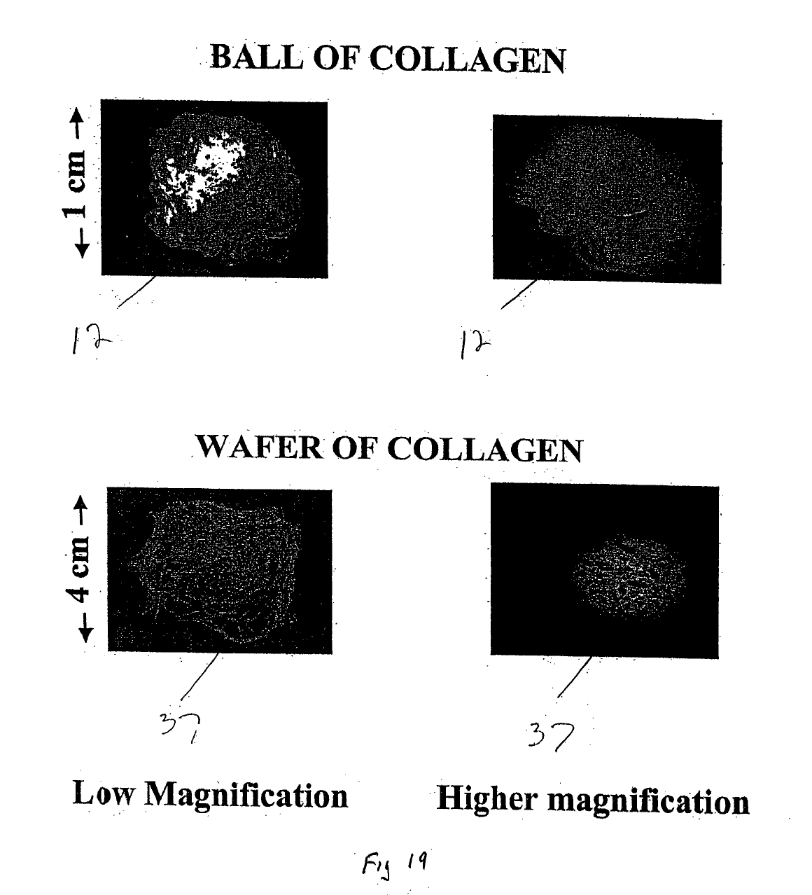

In one embodiment, the microparticulate support material comprises one or more threads. A portion of one thread is as shown in FIG. 1. In the embodiment shown in FIG. 1, thread 12 has cells 11 adhered to the surface of thread 12. In another embodiment, thread 12 can be made of any biodegradable material such as collagen, more specifically type I, type II, or type III collagen, or a combination thereof or one or more of the microparticulate support materials described below. The support materials can also be cross-linked to each other. Typically, the dimensions of thread 12 are suitable for attachment and/or growth of mammalian cells thereon or therein (depending on the porosity of thread 12 and the size of cells 11). Thread 12 is typically about 20 to 400 microns in diameter. In some embodiments of thread 12, the pores have a sufficient diameter to permit migration of cells, such as chondrocyte cells, into the interior of thread 12. Thread 12 can then be used to form a sponge-like material as shown in FIGS. 6 and 7 and described in more detail below or thread 12 can be formed, pressed or rolled into balls of thread 12 (referenced as ball of thread 12A, as shown in FIG. 12) and in some embodiments, thread 12 can have a total surface area of about 30 cm². Thread 12 can also be formed into wafers, as shown in FIG. 19, and also described in more detail below.

In one embodiment, thread 12 can be made by injecting a biodegradable material such as a collagen gel through a capillary tube into a coagulation bath. The thread becomes insoluble after cross-linking occurs. As shown in FIGS. 10, 11 and 12, a solution of oxidized collagen 13, preferably 1% oxidized collagen, can be injected into a bath of cross-linking buffer 14, typically by a needle 16 having a 90° end and 0.5 mm diameter. In one embodiment, the collagen is injected in a continuous or semi-continuous stream or thread 12. As the collagen contacts the cross-linking buffer, the collagen begins to solidify. The ball of thread 12 (depicted in FIG. 12 as 12A) can be formed from a single collagen thread which cross-links to itself or separately cross-linked collagen threads or thread fragments, which can themselves be cross-linked together.

An example of a ball of thread 12 in the form of cross-linked collagen thread is shown in the microscopic view presented FIG. 19.

Alternatively, in one embodiment in which threads are used as a support material, threads 12 can be molded, rolled or pressed into other ball-like shapes or formed into a sponge-like material, as described below.

B. Beads

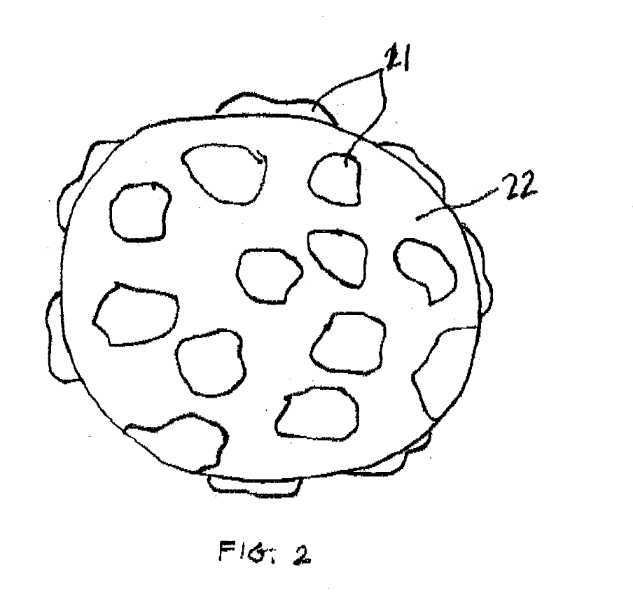

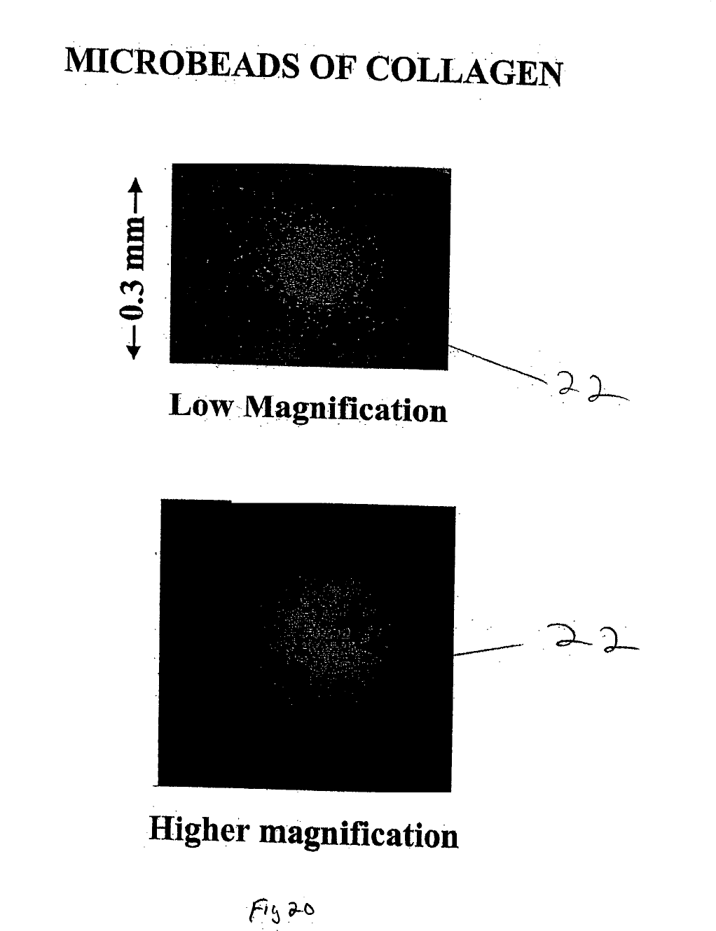

In another embodiment, the microparticulate support material includes one or more beads 22, shown in FIG. 2. In the embodiment shown in FIG. 2, bead 22 has cells 11 attached and/or grown into the surface of bead 22 (depending on the porosity of bead 22 and the size of cells 11). Bead 22 can be made of any biodegradable material including type I, type II, or type III collagen or a combination thereof, or one or more of the microparticulate support materials described below. Typically, the diameter of bead 22 is a size that is suitable for attachment and/or growth of mammalian cells thereon, usually from 20 to 400 microns in diameter. An example of a bead 22 formed of cross-linked collagen is shown in the microscopic view presented in FIG. 20. In another embodiment, if bead 22 has sufficient porosity, cells 11 can be attached and/or grown within bead 22. In a porous embodiment of bead 22, the pores have a sufficient diameter to permit migration of cells, such as chondrocyte cells, into the interior of bead 22.

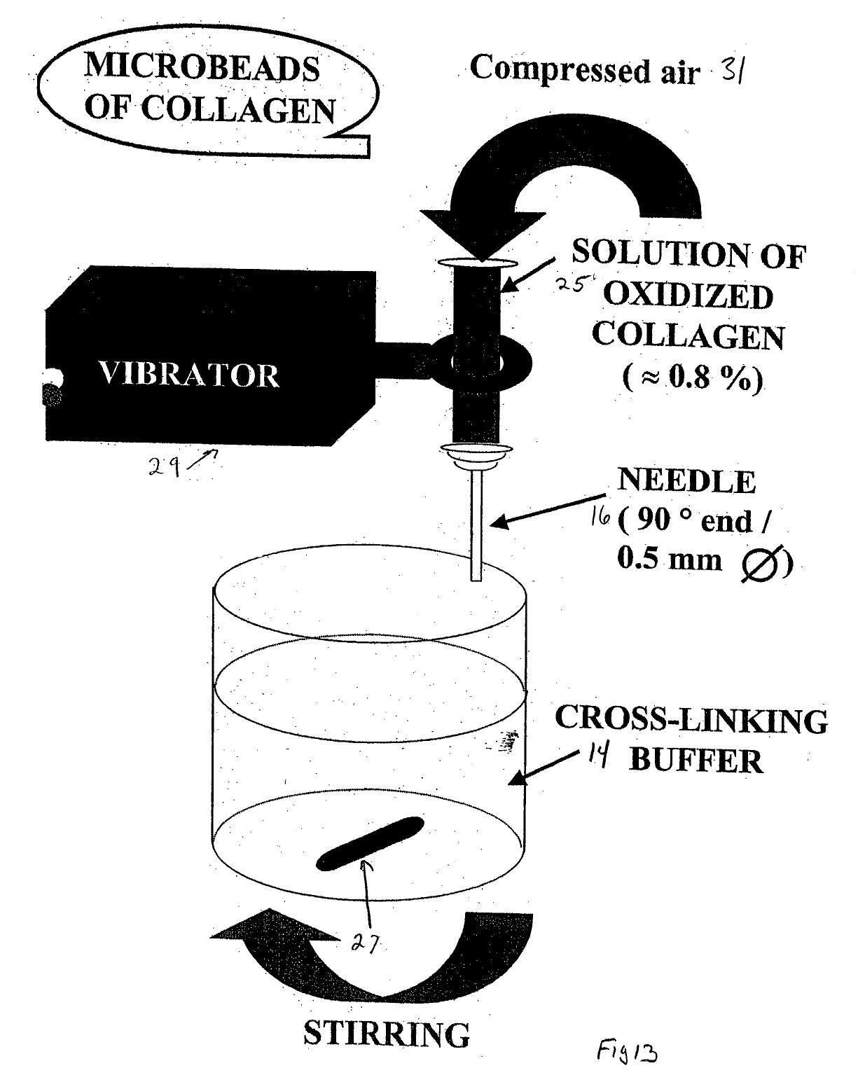

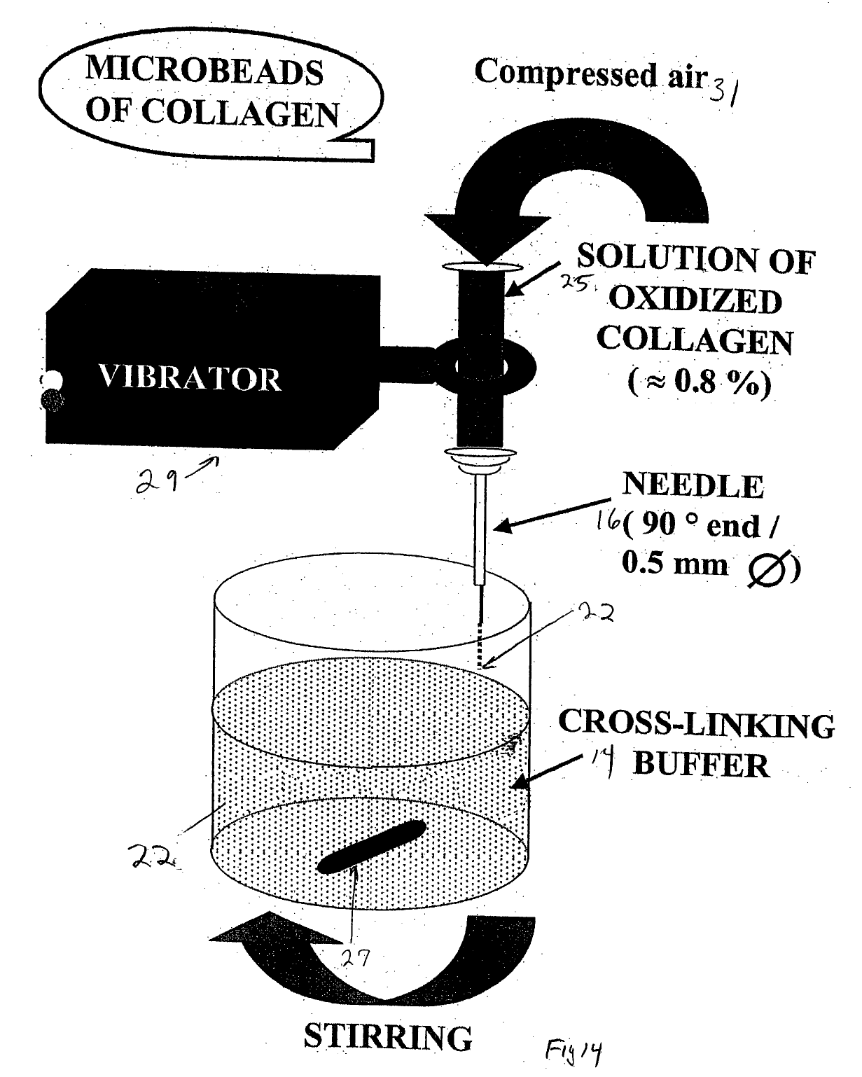

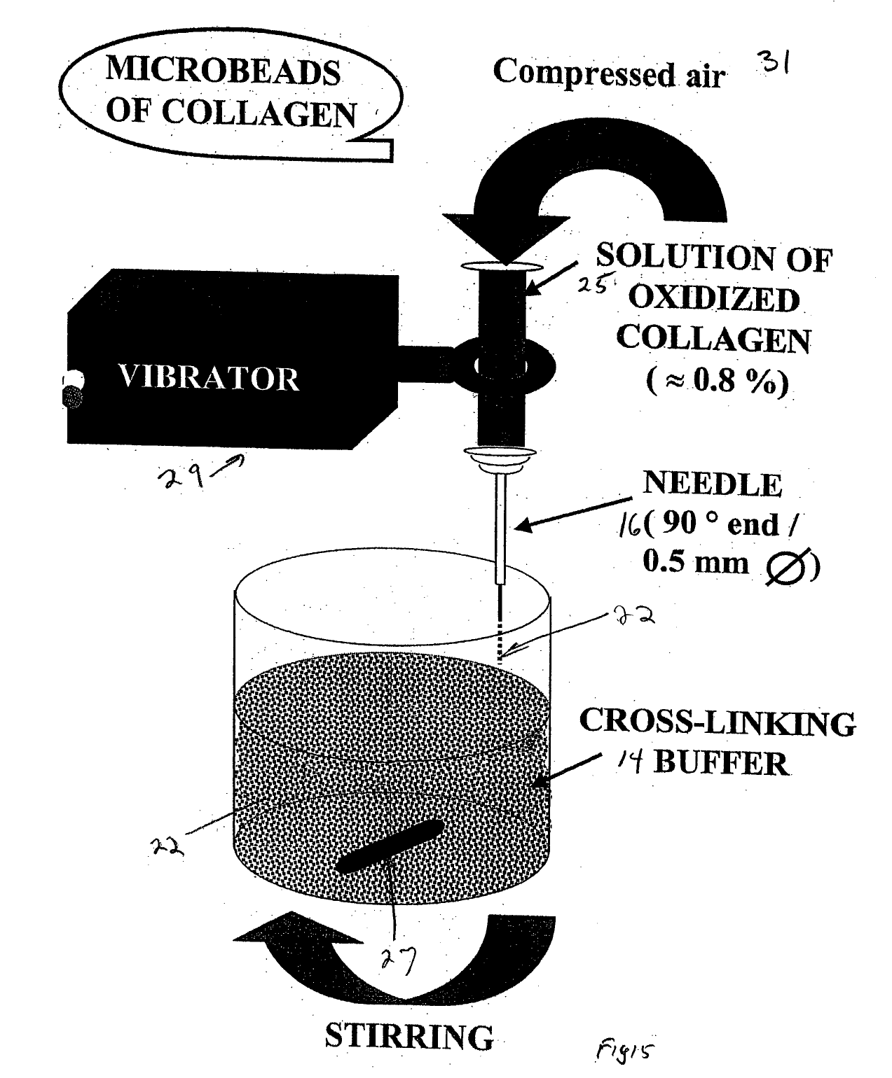

In one embodiment, bead 22 can be made according to process described in EP Patent Publication 351296 A1 to IMEDEX, the entire content of which is hereby incorporated by reference. According to the IMEDEX EP publication, collagen type I droplets from the dermis of either porcine or bovine origin are formed and recovered from a solution of collagen. As shown in FIGS. 13, 14, and 15, a solution of oxidized collagen 25, typically about 0.8% collagen, can be injected by needle 16 into a solution of cross-linking buffer 14 to form beads 22. In one embodiment, compressed air 31 is used to drive the collagen solution into needle 16. As the collagen is injected into the cross-linking buffer, a vibrator 29 gently shakes the injecting device causing the collagen to fall from the needle in a dropwise fashion, forming bead droplets 22. As bead droplets 22 separately contact the cross-linking buffer, the surface of bead droplets 22 cross-link, solidify and collect as solid beads 22 in the cross-linking buffer, as shown in FIGS. 14 and 15. Separation of the beads can be maintained by stirring, and in one embodiment by a magnetized stirring bar 27. The collagen droplets are then separated from the solution as solidified collagen beads 22. In this embodiment, the bead size ranges from a diameter of about 20 microns to about 2 mm. An example microscopic view of the collagen beads of the present invention is shown in FIG. 20.

C. Wafers

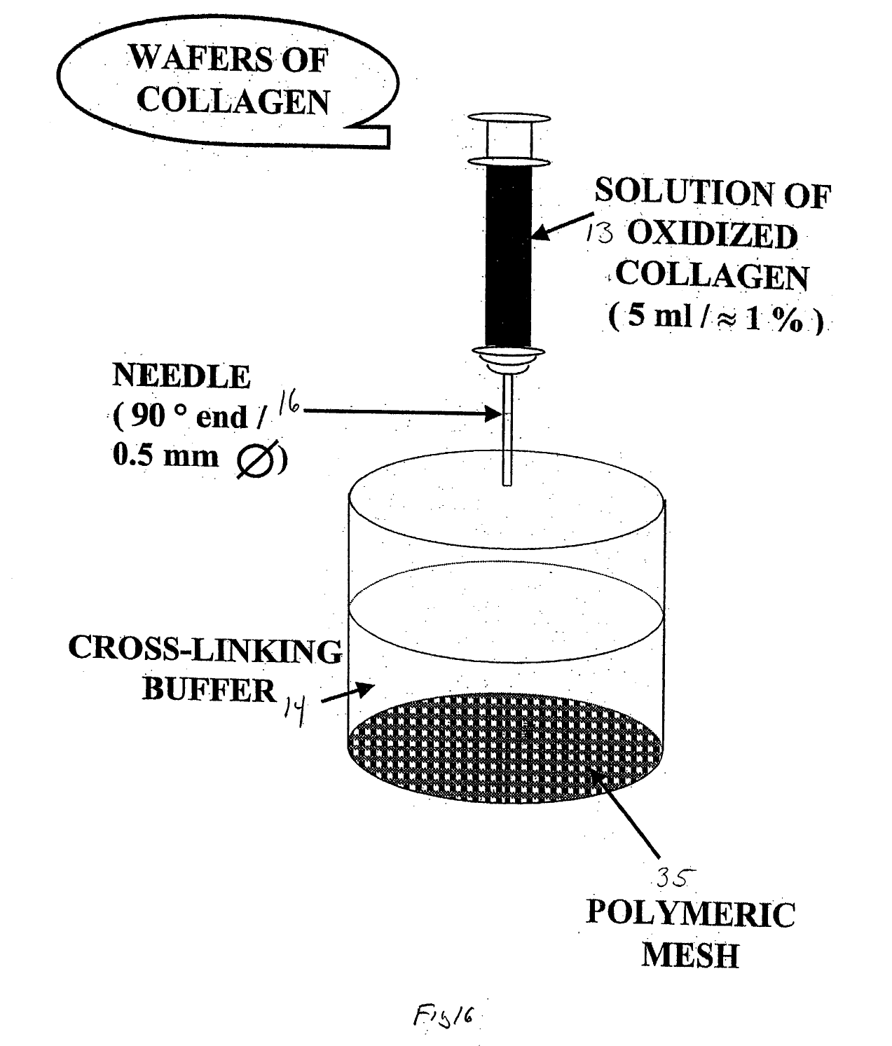

As further shown in FIGS. 16, 17 and 18, a wafer 37 can also be formed by injecting a volume of solution of oxidized collagen 13 by a needle 16 into a cross-linking buffer 14 that optionally contains a polymeric mesh 35 for collagen thread 12 to cross-link and settle thereon. In one embodiment according to this method, a continuous or semi-continuous monofilament of thread 12 can be manually prepared and packed within a three-dimensional volume of any appropriate size before dehydration, thereby forming an alternative embodiment of wafer 37. An example of wafer 37 formed of cross-linked collagen thread is shown in the microscopic view presented FIG. 19.

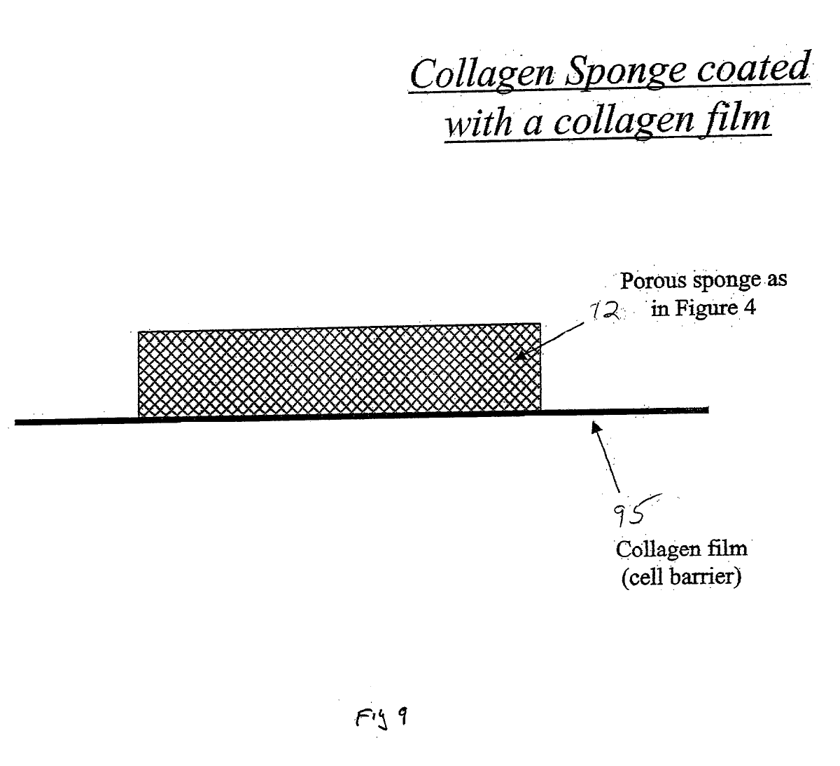

Once formed, wafer 37 (or another microparticulate support material described herein) can be integrated into a film of additional collagen (a cross-linked or un-cross-linked collagen film). In one embodiment, wafer 37 can be integrated into a film of collagen by placing wafer 37 on a collagen film that is contained in a solid support, such as a petri dish. As shown in FIG. 9, in one embodiment threads 12 (after threads 12 are dried and randomly packed in a volume, as shown in FIG. 7) are integrated into film 95, which forms a cell barrier to prevent cell migration.

In some embodiments, wafers 37 have a surface area available for cell culture of up to about 50 cm².

D. Other Forms of the Microparticulate Support Material

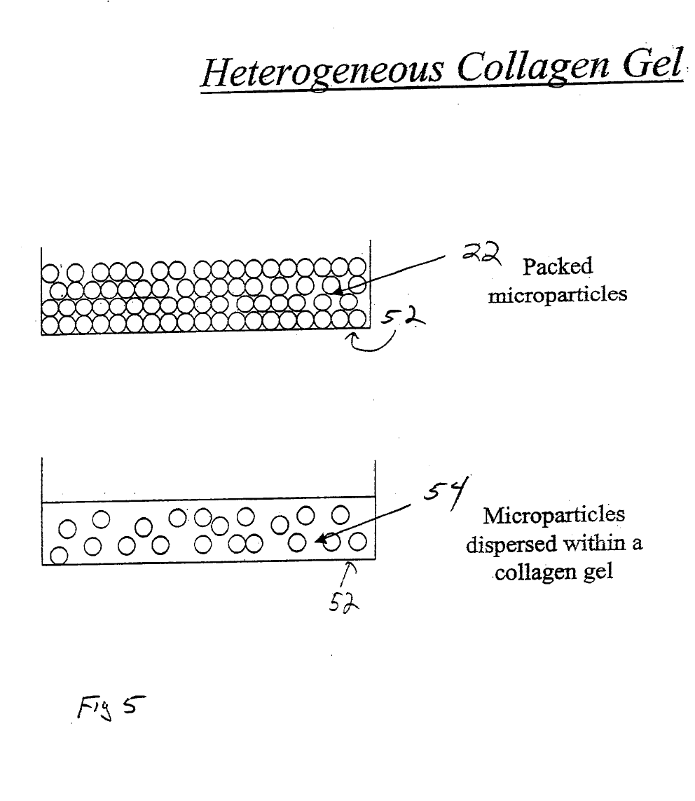

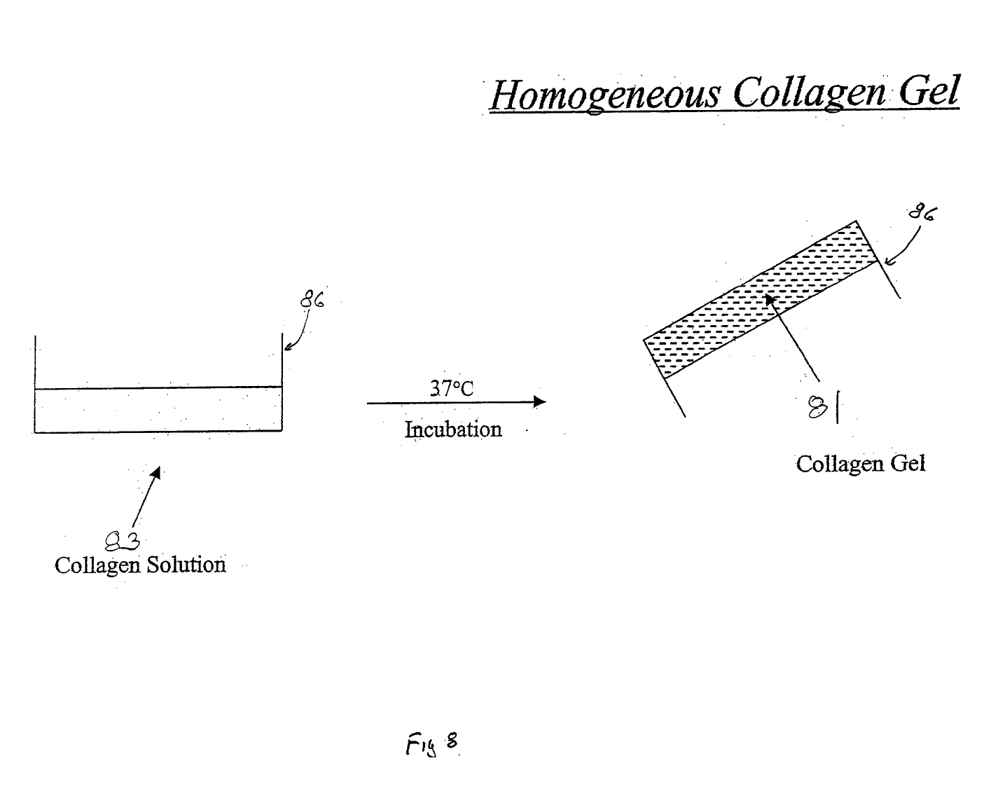

In one embodiment, homogenous collagen gel 85 can be made in the manner depicted in FIG. 8, namely by incubating a solution 83 of the desired support material (such as collagen) in a container, such as depicted container 86. However, in another embodiment, a sponge-like gel structure can be made from beads 22 or threads 12. Specifically, beads 22 and/or threads 12 can be packed together to form the gel structure including beads 22 and/or threads 12 and gel 54, such as that shown in FIGS. 5, 6, 7 and 8. The porosity of the gel structure is defined by the interstitial volume between the particles, which can typically range from 30% to 50% of the total volume of the packed beads 22 and/or threads 12. In one embodiment, gel 54 is collagen.

In FIG. 5, the microparticulate support material includes beads 22 of the present invention, (although the microparticulate support material can also include threads 12 and/or wafers 37), which are packed in a container 52. In one embodiment, beads 22 are then dispersed within a collagen gel 54.

In the embodiment depicted in FIG. 6, the microparticulate support includes a monofilament of thread 12 which is packed into container 62 before or after thread 12 has been cross-linked, and, after drying, forms a dried insoluble monofilament. In one embodiment, thread 12 of the invention can be packed into a container before or during cross-linking such that thread 12 becomes randomly packed and interconnected in container 72, as shown in FIG. 7.

In an alternate embodiment, the microparticulate support material (either thread 12 or bead 22) can be made from one or more other resorbable materials. Such microparticulate support materials can be prepared from alginate, starch, hyaluronan, dextran (See Van Wezel, A. L. 1967, Nature 216:64:65); cellulose (See Reuveny, S., et al., 1982, Dev. Biol. Stand. 50:115-123); collagen (See R. C. Dean et al., 1985, Large Scale Mammalian Cell Culture Technology. Ed. B. K. Lydersen, Hansen Publishers, New York, N.Y., pp. 145-167); or gelatin (See Cultisphere, Technical Bulletin, Percell Biolytica AB), so long as the appropriate dimension criteria are met. Other relevant criteria can include porosity of the support material, degradation time of the support material and whether the support material is cross-linked. In one embodiment, the microparticulate support material of the present invention comprises collagen in combination with one or more other resorbable materials. Further, in accordance with the present invention, the microparticulate support material can be uncross-linked or cross-linked using one or more cross-linking agents apparent to one of skill in the art. An appropriate cross-linking agent includes glutaraldehyde and similar products. Preferably, the microparticulate support material includes a biodegradable material which will support chondrocyte cell attachment and/or growth on or within the microparticulate support material, and which, over time will be absorbed in the body of a patient receiving the implant.

Also, the present invention includes a method of making an implantable composition including adding chondrocyte cells to the microparticulate support material, described above. In such an embodiment the beads, threads or a mixture of beads and threads are prepared according to the present invention. Adherent cells have a natural tendency to adhere to the surface of the microparticulate support material. However, in another embodiment, the method further comprises mixing an adhesive, such as one or more of the adhesives described below, with chondrocyte cells and a microparticulate support material. In embodiments in which an adhesive is used, (1) cells are adhered to the support material with a layer of adhesive applied to the support material before the cells, (2) one or more layers of adhesive are applied over the cells which have been adhered to the support material without an adhesive, or (3) a mixture of adhesive and cells are adhered to the support material. The present invention may also utilize autologous, and/or allogeneic chondrocytes and/or xenogeneic chondrocytes.

In one embodiment, the microparticulate support material can be sterilized by methods apparent to one of skill in the art, typically by beta or gamma irradiation as well as ethylene oxide diffusion.

As used herein, the implantable composition of the present invention includes a microparticulate support material having cells adhered thereto. The implantable composition optionally further includes appropriate excipients and adhesives, as described herein.

### Excipients

It has been found that in some embodiments, separate particles of a microparticulate support material having cells adhered thereto can become associated by forces such as gravity and Van Der Waals forces, thereby forming a sediment at the bottom of a vessel that contains the microparticulate support material and cells (i.e., the implantable composition). This sediment has a range of viscosities and may or may not or may not be flowable, depending upon a number of factors. As used herein, flowable means to move or run smoothly with unbroken continuity, as in the manner characteristic of a fluid. However, in some embodiments the present invention can flow slowly, for example when a viscous gel is used as an excipient, as described below and can therefore range from free flowing to hardened. Factors affecting the flowability of the present invention include 1) the duration of time the microparticulate support material and cells remain in a containment vessel, 2) the dimensions and shape of the individual particles of the microparticulate support material and 3) the amount of cell growth on the microparticulate support material and any surrounding material.

Accordingly, it is oftentimes desirable to adjust the flowability of the above described composition to facilitate administration, for example by injection, of the present invention to a patient. To adjust the flowability of the composition, excipients can be combined directly with the implantable composition. The factors that affect the flowability of an excipient, and thus the flowability of the implantable composition, are appreciated by one of skill in the art, and include but are not limited to, density and viscosity. Other factors that can affect flowability include the chemical and physical characteristics of the adhesive used and further excipients or additives used in the invention. In some embodiments, since the microparticulate support materials are fixed in a defect by glue, viscosity also depends on time point of measurement, and therefore ranges from fluid to fixed.

Suitable excipients include any biocompatible (for example, with chondrocytes and with any tissue in which it may be implanted) liquid, suspension, gel or gel-like material or a microparticulate solid or semisolid material, characterized by the ability to retain chondrocyte cells on the surface or within the surface, for a period of time to enable the attachment and/or growth and/or multiplication of chondrocyte cells therein or thereon, both before implantation and after implantation to a surface to be treated, and to provide a system similar to the natural environment of the chondrocyte cells to optimize cell growth as well as cell differentiation (if applicable to the particular type of cell used). Preferably, the microparticulate support material includes a biodegradable material which will support chondrocyte cell attachment and growth and which, over time will be absorbed in the body of a patient receiving the implant.

### Adhesives

In one embodiment, the implantable composition further includes any biocompatible adhesive. Such adhesives include collagen or fibrin glue, physiological glues, autologous glue, semi-autologous and non-autologous glue or gel. The implantable composition of the present invention optionally further includes one or more of an adhesive and/or excipient, such as a gel, skin glues, surgical glues, and alginates. A specific example of an applicable adhesive includes Tisseel VH™ fibrin sealant, available from Baxter Healthcare Corporation 1627 Lake Cook Road, LC-IV Deerfield, Ill. 60015, USA. Suitable organic glue material can be found commercially, such as for example Tisseel® or Tissucol® (fibrin based adhesive; Immuno AG, Austria), Adhesive Protein (Cat. #A-2707, Sigma Chemical, USA), and Dow Corning Medical Adhesive B (Cat. #895-3, Dow Coming, USA).

As described above, the biocompatible adhesive can be combined directly with the microparticulate support material having cells adhered thereon, thereby affecting the flowability of the present invention, as described in more detail below. Alternatively, the adhesive can be applied in a layer on a surface to be treated followed by a layer of microparticulate support material having cells adhered thereon. Alternatively, the adhesive can be applied in a layer after a layer of microparticulate support material having cells adhered thereon is applied to a surface to be treated. In other embodiments, the biocompatible adhesive, cells and microparticulate support material are applied to a surface to be treated separately or in combination after being mixed.

In an embodiment wherein the adhesive is combined directly with the implantable composition of the present invention, the adhesive also provides the advantage of adjusting the flowability of the present invention to suit the particular needs of a chondrocyte recipient. The adhesive affects the flowability of the present invention in a manner similar to that of the excipients described above, depending on the characteristics of the adhesive, such as viscosity and density.

### Method of Treatment

The present invention provides a method for the effective treatment of articulating joint surface cartilage by the implant of a composition to a surface to be treated by first placing an implantable composition upon a surface to be treated and permitting the chondrocyte cells to attach and proliferate on the surface. The cells then produce a cartilage matrix, and proliferate and populate the cartilage matrix. In another embodiment, the method comprises the additional step of covering the surface to be treated with a covering patch, such as that described in U.S. Pat. No. 5,857,269, the entire content of which is incorporated by reference.

The covering patch may be partially attached to the surface to be treated before placing the implantable composition upon the surface to be treated or placed on the surface after placing the implantable composition upon the surface. The covering patch is capped over the repair site such that the transplanted chondrocytes are held in place, but are still able to gain access to nutrients. In one embodiment, the covering patch is a semi-permeable collagen matrix having at least one porous surface. If used, the covering patch preferably is a cell-free, physiologically absorbable, non-antigenic membrane-like material. In one embodiment of the present invention, a porous surface of the covering patch is directed toward the surface to be treated. Further, in one embodiment the covering patch is in a sheet like form having one relatively smooth side and one relatively rough porous side. In this embodiment, the rough porous side typically faces the cartilage defect and promotes chondrocyte cell in-growth, while the smooth side typically faces away from the cartilage defect and impedes tissue in-growth. In another embodiment, the covering patch has two smooth sides of similar porosity.

Two materials suitable for use as covering patches include Chondro-Gide® or Bio-Gide®, commercially available type I/typeII collagen membranes (Ed. Geistlich Sohne, Wolhusen Switzerland). Additional material that can be used in accordance with the present invention is Chondro-Cell®, a commercially available type II collagen matrix membrane (Ed. Geistlich Sohne, Switzerland).

### Hemostatic Embodiments

In one embodiment, the methods of the present invention also include the use of hemostatic products in conjunction with the transplantation of the implantable composition and, optionally, with a covering patch. Hemostatic products inhibit the formation of vascular tissue, for instance such as capillary loops projecting into the cartilage being established, during the process of autologous transplantation of chondrocytes into defects in the cartilage. Such products are sometimes useful in repairing cartilage defects in bones where the defects extend into or below the subchondral layer, sometimes referred to as a full thickness defect. The formation of vascular tissue from the underlying bone will tend to project into the new cartilage to be formed leading to the appearance of cells other than the mesenchymal specialized chondrocytes desired. The contaminating cells introduced by the vascularization may give rise to encroachment and over-growth into the cartilage to be formed by the implanted chondrocytes.

Although the present invention can be used in conjunction with a hemostatic product or barrier, it has been found that in certain embodiments where an adhesive or excipient is used with the implantable composition of the present invention, such as those described above, the adhesive or excipient can function as an effective hemostatic barrier. However, in another embodiment, an optional membrane such as those described above, can be used to prevent blood from contacting the implantantable composition.

One of the types of commercial membrane products which can be used in accordance with this invention is Surgicel® (Ethicon Ltd., UK), which is absorbable after a period of 7-14 days. This is contrary to the normal use of this particular hemostatic device, such as Surgicel®, as described in a product insert from Ethicon Ltd. Other membrane products include Chondro-Gide® and Bio-Gide®, described above.

To inhibit the re-vascularization into cartilage, a hemostatic material can be used and will act as a gel like artificial coagulate. If red blood cells should be present within a full-thickness defect of articular cartilage that is covered by such a hemostatic barrier, these blood cells will be chemically changed to hematin and thus not be able to induce vascular growth. Thus, a hemostatic product used as a re-vascularization inhibitory barrier with or without fibrin adhesives, such as for example the Surgicel®, is effective for one embodiment of the methods as taught by the instant invention.

The implantation procedure according to the present invention can be performed by an arthroscopic, miniarthrotomic, or open surgical technique.

It is understood that the defect or injury can be treated directly, enlarged slightly or sculpted by surgical procedure prior to implant such as described in U.S. patent application Ser. No. 09/320,246, the entire contents of which are incorporated herein by reference, to accommodate the implantable composition.

### Culturing Procedures

It is notable that adherent cells 11 and 21 have an optimal surface area upon which to attach and proliferate. If the surface area of bead 22, wafer 37 or thread 12 is too large or too small relative to cells (e.g. cells 21 or 11), then the cells will not grow. Thus, an optimal surface area of microparticulate support material beads 22 or thread 12 relative to cells 21 and 11 for attachment and growth of cells 21 and 11 to bead 22 or thread 12 is necessary. A typical optimal surface area may be achieved by using bead 22 or thread 12 having diameters of 20 microns to 400 microns in diameter.

By way of example, the culturing procedure, the attachment and/or growth of chondrocytes and the transplant media used in the culturing procedure and/or attachment and/or growth of chondrocytes are each described in detail below, starting first with a description of a laboratory procedure used to process the harvested cartilage biopsy and to culture the chondrocyte cells according to the present invention.

### EXAMPLE 1

### Chondrocyte Harvesting and Growth

Growth media (hereinafter, “the growth media”) used to transport and/or process the cartilage biopsy during the culturing process and to grow the cartilage chondrocyte cells is prepared by mixing together 2.5 ml gentomycin sulfate (concentration 70 micromole/liter), 4.0 ml amphotericin (concentration 2.2 micromole/liter; tradename Fungizone®, an antifungal available from Squibb), 15 ml 1-ascorbic acid (300 micromole/liter), 100 ml fetal calf serum (final concentration 20%), and the remainder DMEM/F 12 media to produce about 400 ml of growth media. (The same growth media is also used to transport the cartilage biopsy from the hospital to the laboratory in which the chondrocyte cells are extracted and multiplied.)

For an autologous implant, a cartilage biopsy first is harvested by arthroscopic technique, for example, from a non-weight bearing area in a joint of the patient and transported to the laboratory in a growth media containing 20% fetal calf serum. The cartilage biopsy is then treated with an enzyme such as trypsin ethylene diamine tetra acetic acid (EDTA), a proteolytic enzyme and binding agent, to isolate and extract cartilage chondrocyte cells from the cartilage. The extracted chondrocyte cells are then cultured in the growth media from an initial cell count of about 50,000 cells to a final count of about 20 million chondrocyte cells or more.

Blood obtained from the patient is centrifuged at approximately 3,000 rpm to separate the blood serum from other blood constituents. The separated blood serum is saved and used at a later stage of the culturing process and transplant procedure.

Cartilage biopsy previously harvested from a patient for autologous transplantation is shipped in the growth media described above to the laboratory where it will be cultured. The growth media is decanted to separate out the cartilage biopsy, and discarded upon arrival at the laboratory. The cartilage biopsy is then washed in plain DMEM/F 12 at least three times to remove the film of fetal calf serum on the cartilage biopsy.

The cartilage biopsy is then washed in a composition which includes the growth media described above, to which 28 ml trypsin EDTA (concentration 0.055) has been added. In this composition, the cartilage biopsy is incubated for five to ten minutes at 37° C., and 5% CO₂. After incubation, the cartilage biopsy is washed two to three times in the growth media, to cleanse the biopsy of any of the trypsin enzyme. The cartilage is then weighed. Typically, the minimum amount of cartilage required to grow cartilage chondrocyte cells is about 80-100 mg. A somewhat larger amount, such as 200 to 300 mg, is preferred. After weighing, the cartilage is placed in a mixture of 2 ml collagenase (concentration 5000 enzymatic units; a digestive enzyme) in approximately 50 ml plain DMEM/F12 media, and minced to allow the enzyme to partially digest the cartilage. After mincing, the minced cartilage is transferred into a bottle using a funnel and approximately 50 ml of the collagenase and plain DMEM/F12 mixture is added to the bottle. The minced cartilage is then incubated for 17 to 21 hours at 37° C., and 5% CO₂.

In one embodiment, the incubated minced cartilage is then strained using 40 μm mesh, centrifuged (at 1054 rpm, or 200 times gravity) for 10 minutes, and washed twice with growth media. The chondrocyte cells are then counted to determine their viability, following which the chondrocyte cells are incubated in the growth media for at least two weeks at 37° C., and 5% CO₂, during which time the growth media was changed three to four times.

The chondrocyte cells are then removed by trypsinization and centrifugation from the growth media, and transferred to a transplant media containing 1.25 ml gentomycin sulfate (concentration 70 micromole/liter), 2.0 ml amphotericin (concentration 2.2 micromole/liter; tradename Fungizone®, an antifungal available from Squibb), 7.5 ml 1-ascorbic acid (300 micromole/liter), 25 ml autologous blood serum (final concentration 10%), and the remainder DMEM/F 12 media to produce about 300 ml of transplant media.

### EXAMPLE 2

### Implantable Composition

A support material of choice, for example biocompatible, resorbable beads, mesh or threads, is mixed into a transplant media in a sterile petri dish to “wet” the support material with the transplant media, and in one embodiment the support material can contact the transplant media for 1 to 10 hours or more. The chondrocyte cells are then added to the support material transplant media mixture. The transplant media may be 20% minimal essential culture medium containing HAM F12 and 15 mM Hepes buffer and 10 to 20% autologous serum, all of which are contained in a CO₂ incubator at 37° C.

The chondrocyte cells are then allowed to attach and grow on or in the support material for a period of time, ranging from one hour to one week, and in one embodiment the chondrocyte cells are maintained at a temperature of about 37° C. Preferably, the chondrocyte cells are cultured with the support material overnight. In one embodiment, the chondrocyte cells and media are gently stirred to allow the chondrocyte cells to adhere to and grow on all sides of the support material.

In an alternate embodiment, additional support material is added to the chondrocyte cell and support material culture during stirring to allow for the additional attachment and growth of chondrocyte cells on the newly added support material. In some embodiments between 10 and 40 mg. of support material can be added to the culture. In other embodiments, the addition of support material can be repeated from 1 to 20 times. Once the culture period is complete, which can last from about one day to about six weeks, the media containing chondrocyte cells adhered on or within the support material, is ready for placement (for example, by injection) into a defect site.

In another embodiment, during the culture period the support material can be enzymatically dissolved (using, e.g., collagenase), thereby releasing the cells. The enzyme can then be removed from the culture and additional support material can be added to the cell culture.

The support material added in subsequent steps can be the same type of support material or it can be a different type of support material. In one embodiment, the cells can be transferred from a smaller (20 μm) to a larger (400 μm) support material. In another embodiment, the cells can be transferred from a larger to a smaller support material. In yet another embodiment, the cells can be transferred from beads to threads to wafers or any combination thereof having the appropriate size and surface area to facilitate cell growth. The transferring step can be repeated from 1 to 20 times.

### EXAMPLE 3

### Implantable Composition

In another embodiment, chondrocyte cells suspended in the media may be added directly to the support material, without “pre-wetting” of the support material. In this case, the chondrocyte cells are then allowed to attach and grow on or in the support material for a period of time, ranging from about one hour to about six weeks. Preferably, the chondrocyte cells are cultured with the support material overnight. In one embodiment, the chondrocyte cells and media are gently stirred to allow the chondrocyte cells to adhere to and grow on all sides of the support material.

In an alternate embodiment, additional support material (either the same or different support material as the original support material) was added over the culture period to expand the cell culture. In another embodiment, the support material was first destroyed by using enzymes such as trypsin. Then, additional support material having a larger surface area than the original support material that was destroyed, is added to the cell culture. The process of destroying the support material can be repeated two or more times over the culture period.

Once the culture period was complete, the media containing chondrocyte cells adhered to and grown on or in the support material, is ready for placement into a defect site.

### EXAMPLE 4

### Testing of Alternative Support Materials

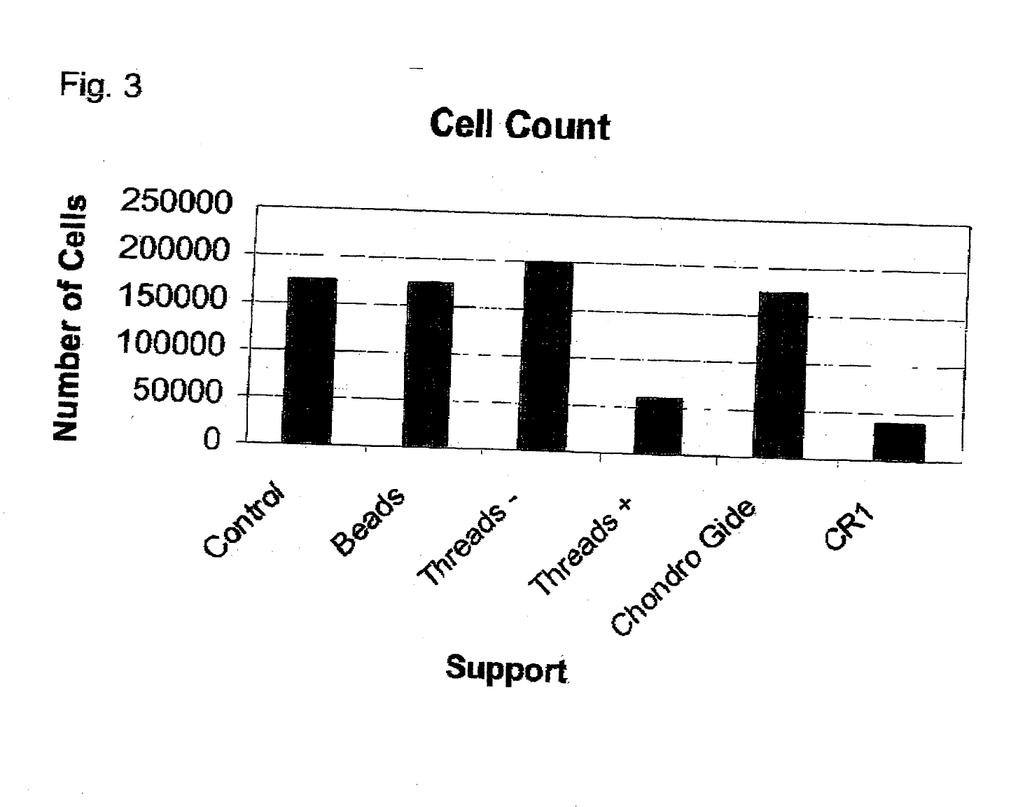

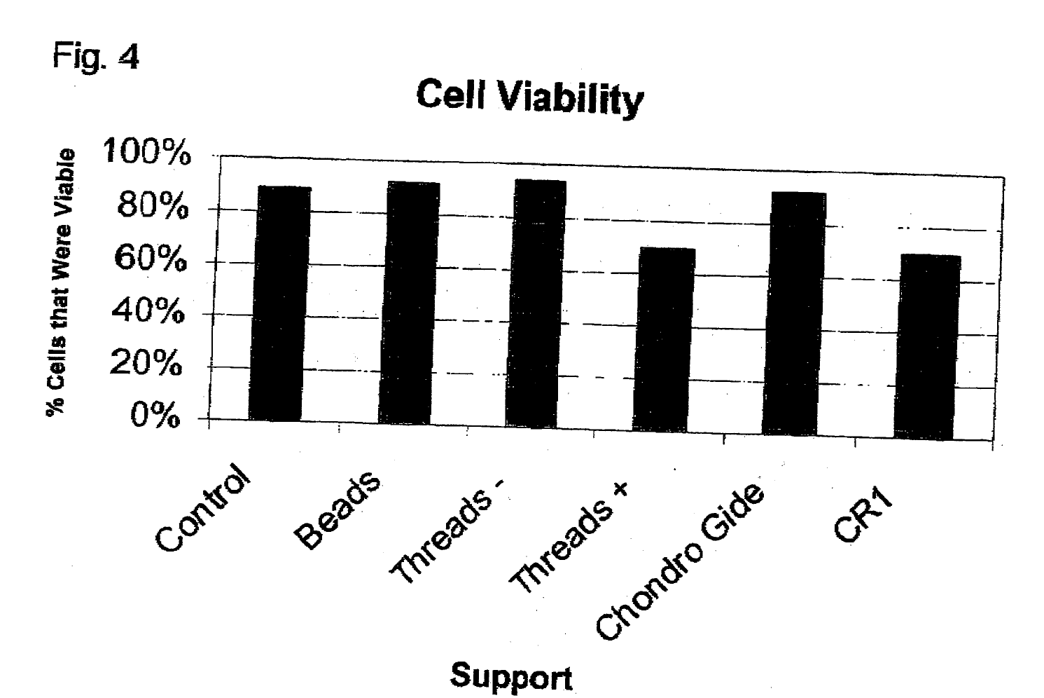

Different support materials were tested for their ability to provide support for cell attachment and growth, as well as cell viability. The following support materials were tested: collagen threads from IMEDEX (not yet commercially available) cross-linked with glutaraldehyde (identified as “Threads+” in FIGS. 3 and 4); collagen threads cross-linked without glutaraldehyde (identified as “Threads−” in FIGS. 3 and 4) as described in the published IMEDEX Patent Publication 351,296 A1 described above; and beads of collagen cross-linked without glutaraldehyde (identified as “Beads” in FIGS. 3 and 4). A Chondro-Gide® membrane (as a positive control), CR-1, an IMEDEX® membrane, and no membrane (as a negative control) were also tested as comparative support materials.

The collagen threads were pressed to form round irregular shapes (for example balls of thread of a globular shape), roughly having diameters of about 0.5 cm. Even though the threads were pressed to form a globular shape, they are referred to herein as “threads.” Samples of the beads and both thread types were weighed under sterile conditions and placed into a 12-well plate. The weight of these samples is shown in the Table 1 with the respective experimental run number.

TABLE 1
Weight carrier in each sample (mg)

| | | Threads | Threads |
|---|---|---|---|
| | | Cross-Linked Without | Cross-Linked With |
| Run # | Beads | Glutaraldehyde | Glutaraldehyde |
| 1 | 43.5 | 42.7 | 41.7 |
| 2 | 35.8 | 39.8 | 39.5 |
| 3 | 42.4 | 44.3 | 40.2 |
| 4 | 48.6 | 42 | 39.2 |
| 5 | 47.3 | 41.7 | 39.1 |
| 6 | 39.5 | 36.3 | 38.6 |
| 7 | 36.5 | 35.5 | 27 |
| 8 | 37.8 | 45.3 | 44 |
| 9 | 40.3 | 27.3 | 35.9 |
| 10 | 39 | 44 | |
| 11 | 40 | 44 | |
| 12 | 40 | | |

The materials were then washed with phosphate-buffered saline (PBS) and the pH of the wash solution checked to determine if the support material caused a change in the pH value. Media (DMEM+20% fetal calf serum (FCS)) was added to each of the wells containing support materials and to the empty well (as shown in FIGS. 3 and 4). A chondrocyte cell suspension was prepared in accordance with routine cell culture techniques and added to the control well and the wells containing the support materials. The chondrocytes were incubated with the support materials for three days at 37° C. in the CO₂ incubator. After three days, the media was removed from the wells and the support materials were washed with PBS.

Next an enzyme solution (0.25% of trypsin, 5,000 U/ml of collagenase) was added to each well and incubated at 37° C. in order to dissolve the support material. The dissolution of each sample was microscopically determined and upon dissolution the suspension within the well was transferred to a centrifuge tube. The wells were then rinsed with DMEM and transferred to the centrifuge tube. The suspensions were centrifuged for ten minutes at 200×g and the supernatant was then discarded. The remaining pellet was resuspended in 0.50 ml of DMEM and the cells counted.

As shown in Table 1, a total of 12 runs were performed on the beads, 11 runs on the threads without glutaraldehyde and 9 runs on the threads with glutaraldehyde. The results of the test for cell number and viability were then averaged and set forth below and in FIGS. 3 and 4.

The results indicated that the highest amount of cells were harvested from the threads cross-linked without glutaraldehyde (“Threads−”), as shown in FIG. 3. The amount of cells on the beads were equivalent with the negative control and positive control (Chondro-Gide® membrane), and the amount of cells on the threads cross-linked with glutaraldehyde (“Threads+”) and the CR-1 membrane was substantially less. In addition, the results indicate that the viability of the chondrocytes grown on the beads, cross-linked threads without glutaraldehyde and the Chondro-Gide® membrane were equivalent to the negative control chondrocytes, as shown in FIG. 4. The viability of the chondrocytes grown on the threads cross-linked with glutaraldehyde and the membrane was again less than the chondrocytes grown on the other support materials, but in all cases some chondrocyte retention and growth was observed.

More specifically, and as shown in FIG. 3, differences in the amount of cells harvested from different carrier materials was apparent. The largest amounts of cells were obtained from the threads cross-linked without glutaraldehyde (“Threads−”). Approximately equal amounts were obtained from the beads and in the positive and negative control experiments. A smaller number of cells were harvested from the wells in which threads cross linked with glutaraldehyde (“Threads+”) or the CR-1 membrane was the carrier.

FIG. 4 provides the viability of the harvested cells. The threads cross-linked without glutaraldehyde (“Threads−”) resulted in cells having the maximum viability of 94%, on average. The positive and negative control experiment and the beads showed similar results, 92%, 89% and 92% cell viability, respectively. The viability of the cells harvested from the threads cross-linked with glutaraldehyde (“Threads+”) or on the CR-1 membrane was reduced to approximately 70% in both cases.

Additionally, to determine the effect of mechanical reduction of the size of the threads on cell viability, the threads cross-linked with glutaraldehyde were mechanically reduced in size. In this experiment, on average the viability of the cells that were harvested from the threads that were not mechanically reduced was 65%, and the averaged viability of the cells from the mechanically reduced threads was 78%.

This comparative study demonstrates the ability of microparticulate support material in the form of collagen beads and threads generally, and such beads and threads cross-linked without glutaraldehyde particularly, to support the attachment and growth of chondrocytes. The collagen bead -chondrocyte composition and collagen thread-chondrocyte composition, in each case, comprised a flowable composition suitable for chondrocyte implantation.

### EXAMPLE 5

### Adding Support Material

In another example with collagen microbeads, three samples of 40 mg. of microbeads having a surface area of about 0.3 mm² per bead were prepared in the manners described above. One hundred thousand chondrocyte cells were added to each of the samples. The samples were cultured in growth medium for three days in the manner described above. To the first sample, 10 additional mg. of microbeads were added. To the second sample, 20 mg. of microbeads were added. To the third sample, 40 mg. of microbeads were added to the sample. The samples were further cultured for four additional days at 37° C. After four days, to the first sample, 10 additional mg. of microbeads were added. To the second sample, 20 mg. of microbeads were added. To the third sample, 40 mg. of microbeads were added to the sample. The samples were then cultivated for one day.

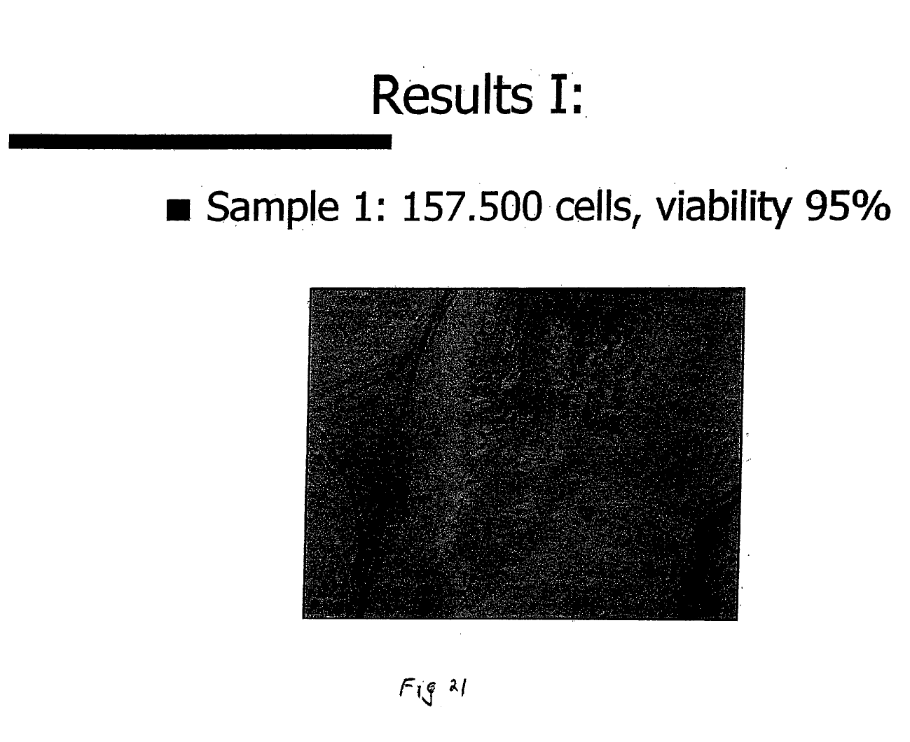

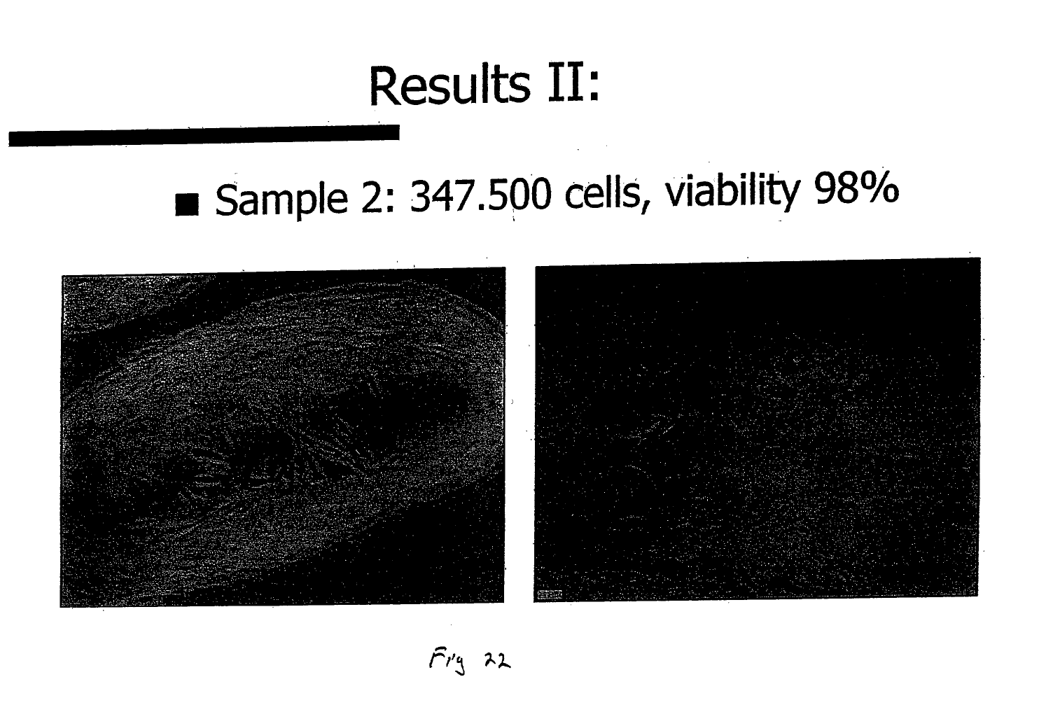

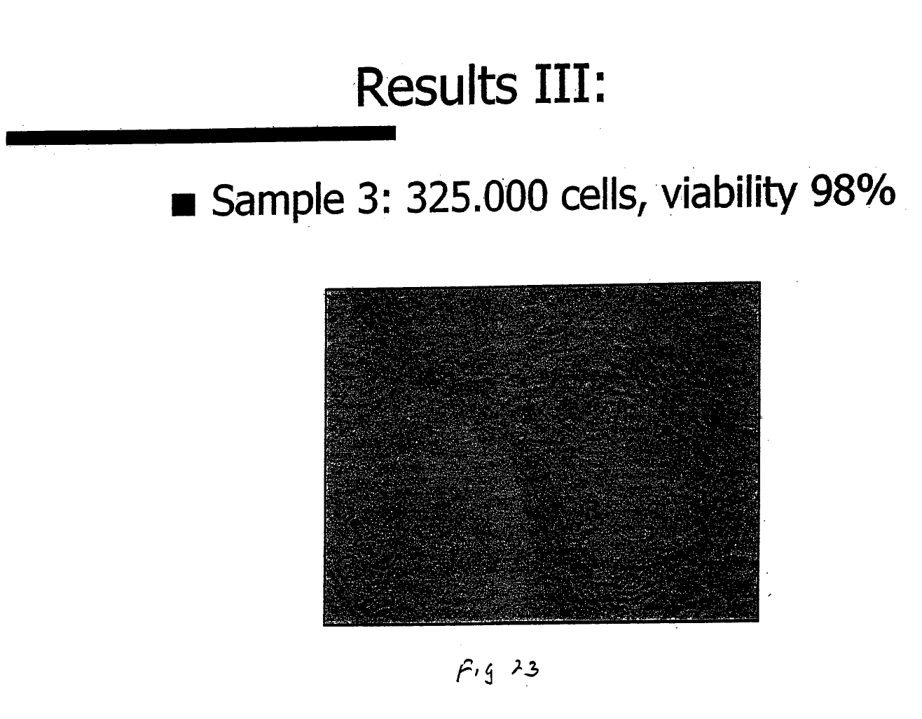

The cells were then counted yielding 157,500, with 95% viability in the first sample (shown in FIG. 21), 347,500 cells in the second sample with 98% viability (shown in FIG. 22), and 325,000 cells in the third sample with 98% viability (shown in FIG. 23). This example indicates the viability of cells cultured on support materials, and the proliferation of cells after adding additional support material.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An implantable composition which is flowable and which comprises chondrocytes retained adjacent a support material.

2. The implantable composition of claim 1 further comprising an adhesive.

3. The implantable composition of claim 1 wherein said support material is a microparticulate solid or semi-solid material.

4. The implantable composition of claim 1 wherein said support material is biodegradable.

5. The implantable composition of claim 4 wherein said biodegradable material is collagen.

6. The implantable composition of claim 5 wherein said biodegradable material is a cross-linked collagen.

7. The implantable composition of claim 1 wherein said chondrocytes are retained on said support material.

8. The implantable composition of claim 1, wherein said chondrocytes are retained in said support material.

9. A method of making an implantable composition comprising
adding chondrocyte cells to a support material, said support material is configured to retain chondrocyte cells adhered thereto; and
forming a flowable mixture of said chondrocyte cells and said support material.

10. The method of claim 9 wherein said forming step includes mixing an adhesive with said chondrocyte cells and said support material.

11. The method of claim 9 wherein said method further comprises exposing said chondrocyte cells and said support material to environmental conditions facilitating the adherence of said cells to said support material.

12. The method of claim 9 wherein said support material is a microparticulate solid or semi-solid material.

13. The method of claim 9 wherein said support material is biodegradable.

14. The implantable composition of claim 13 wherein said biodegradable material is collagen.

15. The method of claim 9 wherein said chondrocyte cells are retained on said support material.

16. The method of claim 9 wherein said chondrocytes are retained in said support material.

17. A method for the effective treatment of articulating joint surface cartilage by the injection of the implantable composition of claim I onto a surface to be treated, the method comprising the steps of:
(a) placing said implantable composition upon the surface to be treated; and
(b) permitting said chondrocyte cells to grow on said surface and form cartilage.
